(19) United States
(12) Patent Application Publication
(10) Pub. No.: US 2013/0205450 A1
(43) Pub. Date: Aug. 8, 2013

(54) CULTIVATION OF PHOTOSYNTHETIC ORGANISMS

(71) Applicants: Fan Lu, Clemmons, NC (US); Hongwu Feng, Wuxi City (CN)

(72) Inventors: Fan Lu, Clemmons, NC (US); Hongwu Feng, Wuxi City (CN)

(73) Assignee: CHLOR BIOENERGY INC., Jersey City, NJ (US)

(21) Appl. No.: 13/695,687

(22) PCT Filed: May 12, 2011
(86) PCT No.: PCT/US11/36273

§ 371 Date: Apr. 12, 2013

Related U.S. Application Data
(60) Provisional application No. 61/334,120, filed on May 12, 2010.

(51) Int. Cl. C12M 1/00

(52) U.S. Cl. CPC C12M 21/02
USPC 800/298; 435/292.1; 435/257.1; 29/428

(57) ABSTRACT

One aspect of the invention features a photobioreactor including at least one plate held in a frame and having a substantially flat surface, a collection tank for holding photysynthetic culture materials, a tunnel positioned at one end of the plate and arranged to conduct culture materials from the plate to the collection tank, and a pump configured to transfer culture materials from the collection tank back to the surface of the plate. In some instances, the plate is supported in a substantially horizontal orientation. In some implementations, the surface of the plate is water resistant.

### TECHNICAL FIELD

This invention relates to cultivation of photosynthetic organisms, and more particularly to a photobioreactor for achieving such cultivation.

### BACKGROUND

In many cases, photobioreactor design and evaluation can determine the success of cultivation of photosynthetic organisms. Most culture systems can be roughly classified into two categories: open raceways (or open ponds) and enclosed photobioreactors. Many open raceways are made of shallow circular channels ranging in size from 1,000 to 5,000 m², either with clayed surface or lined with plastic sheets. Culture mixing is often provided by paddle wheels to affect circulation of the culture suspension around the raceway.

Closed photobioreactors are usually made of transparent materials and have relatively low culture depth or relatively high culture illuminated surface to culture volume ratios. Most closed photobioreactors can be subdivided into two major groups: tubular photobioreactors and flat-plate photobioreactors. Culture mixing and circulation in a closed photobioreactor is usually provided by either mechanical liquid pumps or aeration. For example, Doucha et al. (1999) reported an inclined thin-layer cultivation system on which algal suspension form a thin layer of 5˜18 mm flowing through the inclined cultivation area. The algal suspension is re-circulated by a continuously operating water pump.

Still, it can be particularly difficult to achieve a consistent high yield of cultured biomass when operating under space and/or financial constraints. Thus, other means of cultivating photosynthetic organisms are sought.

### SUMMARY

One aspect of the invention features a photobioreactor including at least one plate held in a frame and having a substantially flat surface, a collection tank for holding photysynthetic culture materials, a tunnel positioned at one end of the plate and arranged to conduct culture materials from the plate to the collection tank, and a pump configured to transfer culture materials from the collection tank back to the surface of the plate. In some instances, the plate is supported in a substantially horizontal orientation. In some implementations, the surface of the plate is water resistant.

In some cases, the photobioreactor includes a first plate and a second plate, the second plate being positioned at a higher elevation than the first plate. In some implementations, an edge of the second plate partially overlaps the surface of the first plate. Further, in some examples, the pump is configured to transfer the photosynthetic culture materials from the collection tank back to a substantially flat surface of the second plate.

In some cases, the photobioreactor further includes a cover positioned over a portion of the surface of the plate. Further, in some implementations, a portion of the cover is substantially transparent.

In some examples, the plate includes a raised edge configured to maintain a predetermined depth of culture suspension on the surface of the plate. In some instances, the raised edge includes a first portion and a second portion, the first portion having a greater height than the second portion. Further in some cases, the height of the second portion is between about 2 cm and 3 cm.

In some implementations, the photobioreactor includes a series of plates arranged in a cascading sequence, such that culture materials fall from an edge of an upper one of the plates onto a lower one of the plates, the tunnel being positioned at one of end of a lowest plate in the series of plates, and the pump being configured to transfer culture materials from the holding tank back to the surface of a highest plate of the series of plates.

One other aspect of the invention features a method of making a photobioreactor including positioning a plurality plates in an overlapping and cascading arrangement such that the first plate is positioned at a higher elevation than the last plate, providing a tunnel in fluid communication, positioning a tunnel in fluid communication with the last plate, and positioning a pump in fluid communication with the tunnel, the first plate being supported in a substantially horizontal orientation and including a raised edge around a portion of its periphery.

Yet another aspect of the invention features a method of culturing photosynthetic materials including introducing a selected amount of photosynthetic culture material to the surface of a plate of a photobioreactor, and circulating the culture material by collecting the culture material from the plate in a collection tank and operating a pump to transfer the culture material from the collection tank back to the surface of the plate.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a photobioreactor.

FIG. 2 is a front view of the photobioreactor of FIG. 1.

FIG. 3 is a side view of the photobioreactor of FIGS. 1 and 2.

FIG. 4 is a top schematic view of a photobioreactor.

FIG. 5 is a front schematic view of the photobioreactor of FIG. 4.

FIG. 6 is a side schematic view of the photobioreactor of FIGS. 4 and 5.

### DETAILED DESCRIPTION

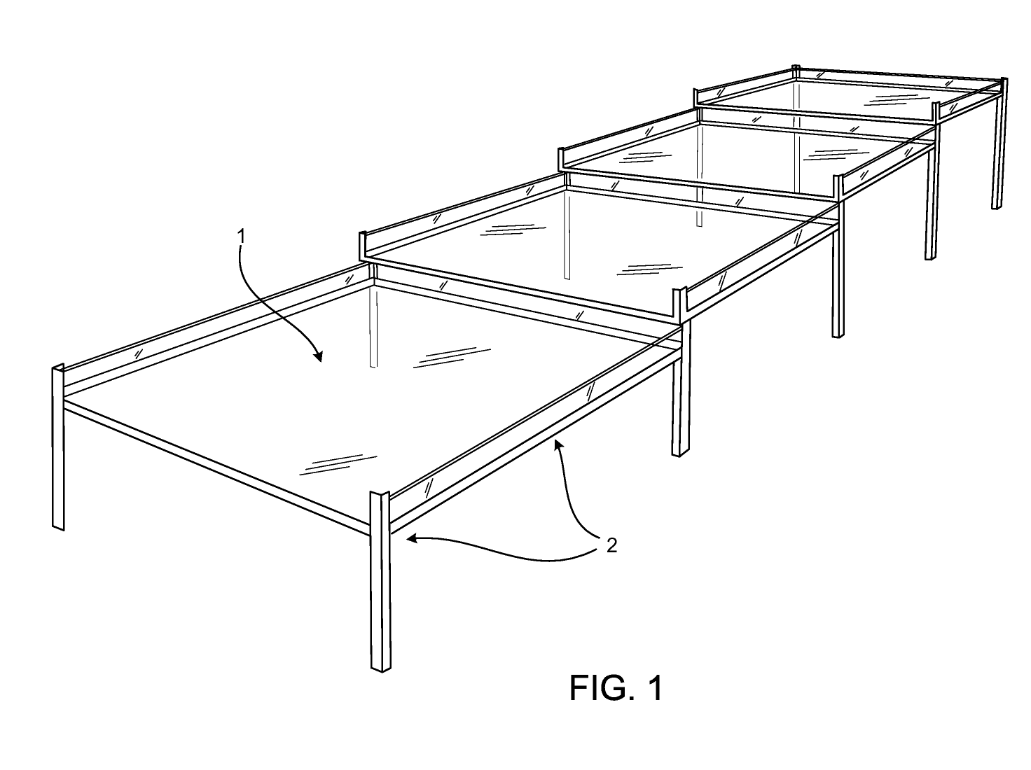

FIG. 1 is a perspective view of a photobioreactor. As shown, the photobioreactor includes a series of horizontally positioned flat plates (1) organized in cascade fashion. The number of flat plates (1) in the cascade can be one, or more than one, depending on the culture requirements and available space. Each flat plate (1) is at a lower position than the preceding flat plate. In this example, an aqueous culture of photosynthetic organisms can be deposited on the highest (first) plate in the cascade, flow across the surface of the plate, and then, by gravity, flow down to the next (lower) plate in the cascade. The culture may be collected at the last flat plate and subsequently be transferred back to the first flat plate by pumping, aeration or other available methods. Such an apparatus can be used for cultivation of any suitable photosynthetic organism (such as algae, cyanobacteria, and plants).

The shape of each individual plate (1) can be square, rectangle, triangle, circle, and alike. The length can range from 30 cm to 1000 m; the width can range from 30 cm to 1000 m. The flat plate (1) can be made of any suitable water resistant materials, such as glass, plastic, cement, brick, metal. Each flat plate is fixed in a supporting frame (2).

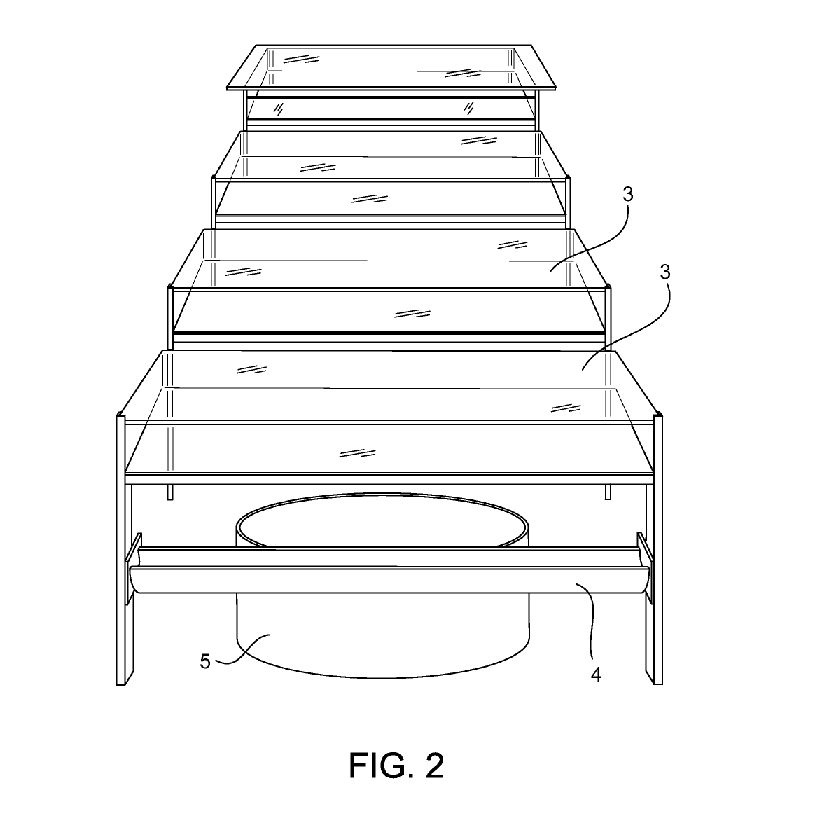

FIG. 2. is a front view of the photobioreactor. As shown, each flat plate is placed horizontally, and is at a lower elevation than the preceding flat plate in the cascade, so that the culture can flow from the upper flat plate to the lower one by gravity . In this example, each plate (1) has a raised edge on three of its sides to inhibit or prevent water overflow (e.g., a 10 cm high raised edge). However, on the outlet side, the height of the raised edge is lower than the other sides (e.g., between about 1 and 3 cm), so that the depth of the culture suspension on the plate will be kept at a desirable level. For example, when cell concentration is high, the depth of the culture can be adjusted to about 1 cm; conversely, if the cell concentration is low, 2 cm or 3 cm culture depth may be desirable to provide more self shading for cells to prevent photoinhibition.

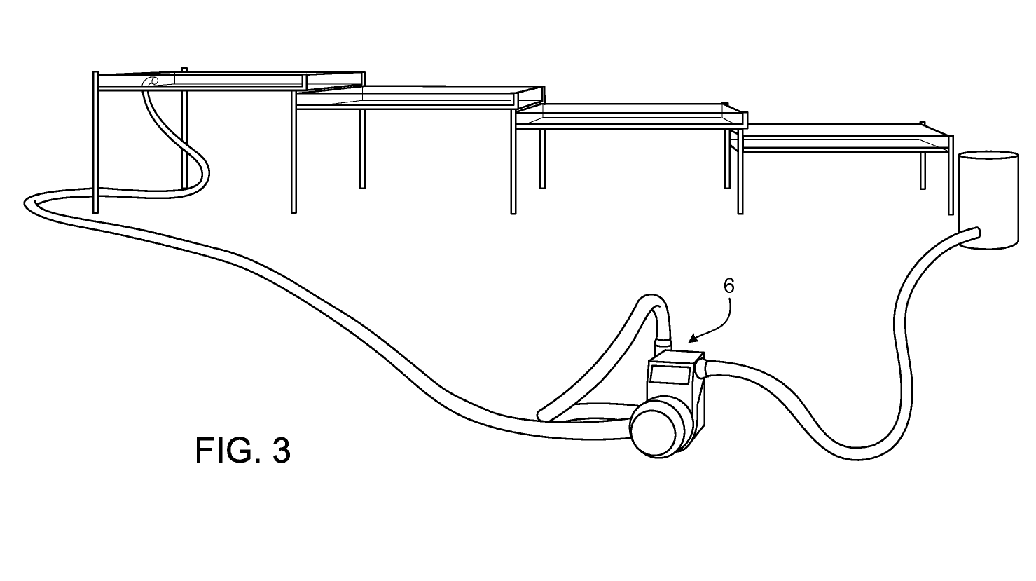

FIG. 3 is a side view of the photobioreactor. As shown, in some examples, the flat plate cascade can be covered by transparent materials (3), such as glass or plastic sheets, to prevent dust and water from contacting the culture.

Referring to FIGS. 2 and 3, in some implementations, at the last flat plate (1), there is a collection tunnel (4) and a reservoir or collection tank (5). Further, as shown in FIG. 3, the culture can be collected at the tank (5) and transferred to the first flat plate (1) in the cascade by a water pump (6).

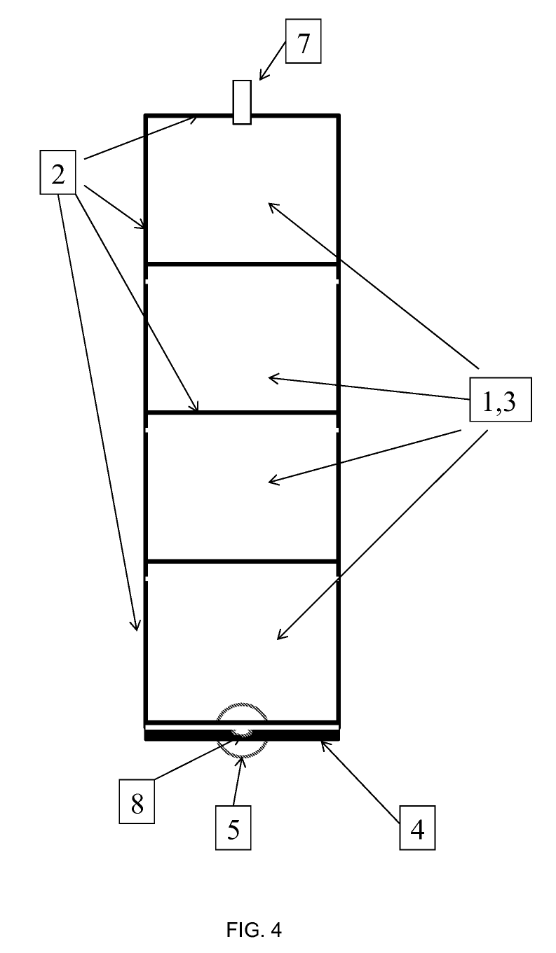

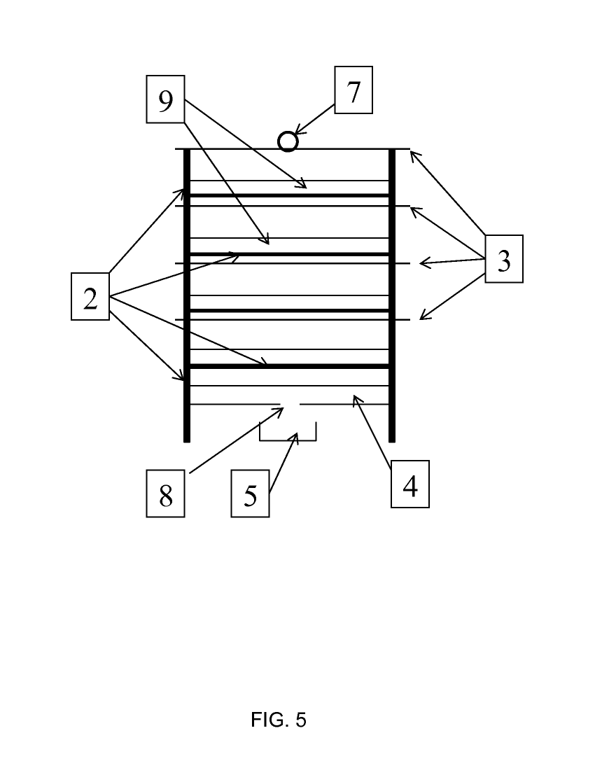

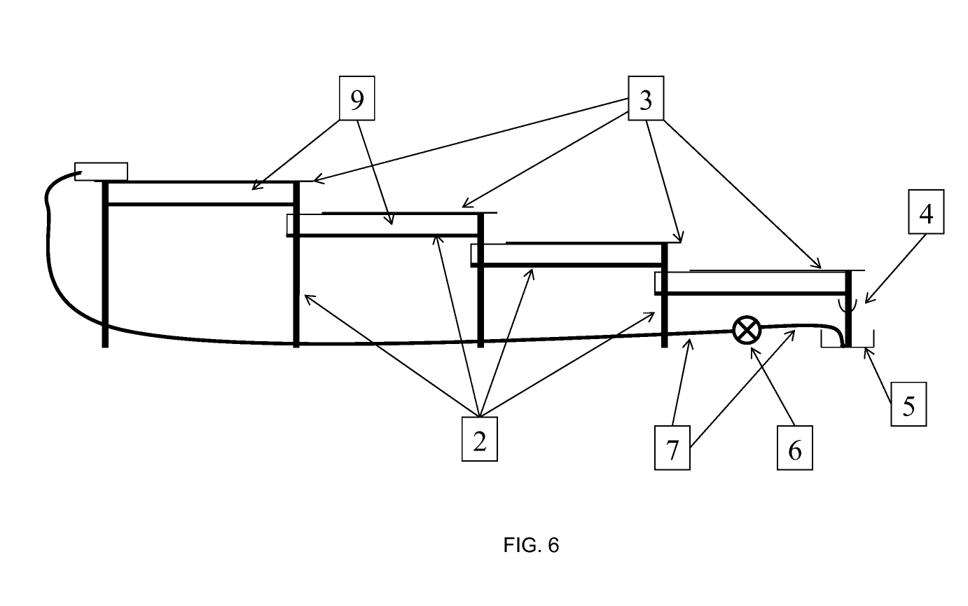

Referring to FIGS. 4-6, in operation, a selected amount of photosynthetic culture material is introduced to the surface of the first plate in the cascading series of plates. The raised edges (9) surrounding the periphery of the plates (1) inhibit overflow of the culture. The culture material flows across the surface of each plate and falls to the surface of the next plate in the series. At the far end of the last flat plate (1) in the cascade, the culture is collected by the tunnel (4) having an orifice (8), through which the culture is conducted to the collection tank (5). The culture is transferred from the collection tank (5) to the first flat plate (1) by a pump (6) and a conveying conduit (7). The transfer can be made by means of any suitable transfer device, such as aeration devices, or mechanical lifting devices. The continuous or semi-continuous transfer of the culture from the collection tank (5) to the first flat plate (1) will keep the culture moving though the flat plate cascade system. In this way, the cultivated cells will be prevented from sedimentation and have an equal opportunity for exposure to sunlight.

In some examples, it is not necessary to circulate the culture suspension continuously. Further, the circulating time of the culture suspension can be controlled by the operation of the water pump (6) at different time intervals, such as 5/15; 5/25; 5/35;5/45, 5/55; etc. (minutes for on/off), depending on the total volume of the culture suspension. In such way, much less electricity will be required to operate the water pump (6).

What is claimed is:

1. A photobioreactor comprising:
at least one plate held in a frame and having a substantially flat surface;
a collection tank for holding photysynthetic culture materials;
a tunnel positioned at one end of the plate and arranged to conduct culture materials from the plate to the collection tank; and
a pump configured to transfer culture materials from the collection tank back to the surface of the plate.

2. The photobioreactor of claim 1, wherein the at least one plate comprises a first plate and a second plate, the second plate being positioned at a higher elevation than the first plate.

3. The photobioreactor of claim 2, wherein an edge of the second plate partially overlaps the surface of the first plate.

4. The photobioreactor of claim 2, wherein the pump is configured to transfer photosynthetic culture materials from the collection tank back to the surface of the second plate.

5. The photobioreactor of claim 1, the at least one plate is supported in a substantially horizontal orientation.

6. The photobioreactor of claim 1, further comprising a cover positioned over a portion of the surface of the at least one plate.

7. The photobioreactor of claim 6, wherein a portion of the cover is substantially transparent.

8. The photobioreactor of claim 1, wherein the at least one plate comprise a raised edge around a portion of its periphery, and wherein the raised edge is configured to maintain a predetermined depth of culture suspension on the surface of the at least one plate.

9. The photobioreactor of claim 8, wherein the raised edge comprises a first portion and a second portion, the first portion having a greater height than the second portion.

10. The photobioreactor of claim 9, wherein the height of the second portion is between about 2 cm and 3 cm.

11. The photobioreactor of claim 1, wherein the at least one plate comprises a series of plates arranged in a cascading sequence, such that culture materials fall from an edge of an upper one of the plates onto a lower one of the plates, wherein the tunnel is positioned at one of end of a lowest plate in the series of plates, and the pump is configured to transfer culture materials from the holding tank back to the surface of a highest plate of the series of plates.

12. The photobioreactor of claim 1, wherein the surface of the at least one plate comprises a water resistant surface.

13. A method of making a photobioreactor, the method comprising:
positioning a plurality of plates in an overlapping and cascading arrangement such that the first plate of the plurality of plates is positioned at a higher elevation than the last plate of the plurality of plates;
positioning a tunnel in fluid communication with the last plate; and
positioning a pump in fluid communication with the tunnel,
wherein the first plate is supported in a substantially horizontal orientation, and
wherein the first plate comprises a raised edge around a portion of its periphery.

14. A method of culturing photosynthetic materials, the method comprising:
introducing a selected amount of photosynthetic culture material to the surface of a plate of a photobioreactor; and
circulating the culture material by collecting the culture material from the plate in a collection tank and operating a pump to transfer the culture material from the collection tank back to the surface of the plate.

15. The method of claim 14, wherein operating the pump comprises activating the pump for a first selected period of time and deactivating the pump for a second selected period of time.
